(19) United States
(12) Patent Application Publication
(10) Pub. No.: US 2003/0215498 A1
(43) Pub. Date: Nov. 20, 2003

(54) Rapidly disintegrating comressed tablets comprising biologically active compounds

(76) Inventor: Ronald S. Harland, Yardley, PA (US)

Correspondence Address: NORRIS MCLAUGHLIN & MARCUS, P.A., P O BOX 1018, SOMERVILLE, NJ, 08876

(21) Appl. No.: 10/150,555

(22) Filed: May 17, 2002

(51) Int. Cl. A61K 9/20

(52) U.S. Cl. 424/465

(57) ABSTRACT

The invention concerns rapidly disintegrating compressed tablets comprising biologically active compounds, preferably having a lipid-based coating and/or a nominal size of up to about 375 microns. The compressed tablets comprise bulking agents having a surface area to volume ratio greater than about 1.0 cm⁻¹. The tablets also comprise binders and lubricants and, optionally, fillers, additives and other excipients. The invention also concerns methods for administering biologically active compounds to human or animal patients via the rapidly disintegrating compressed tablets.

### BACKGROUND OF THE INVENTION

1. Field of the Invention

The invention concerns rapidly disintegrating compressed tablets comprising biologically active compounds preferably having biologically active materials with a lipid-based taste-masking, sustained release, controlled release and/or barrier/protective coating. The compressed tablets further comprise bulking agents having a surface area to volume ratio greater than about 1.0 cm⁻¹, binders and lubricants and, optionally, fillers, additives and other excipients. The tablets are administered orally and will disintegrate within about 40 seconds, or less, after initial exposure to moisture, like bodily fluids, such as saliva. The compressed tablets are also characterized as having improved taste (i.e. masked taste of the active), sustained release, controlled release, acid protection and reduced metabolic or chemical denaturation of the biologically active compound. The invention also concerns methods for administering biologically active compounds to human or animal patients via the rapidly disintegrating compressed tablets.

2. The Prior Art

Easy swallowing dosage forms of biologically active compounds are becoming increasingly demanded, particularly for the aged and children who have difficulty swallowing. Persons having difficulty swallowing may cause capsules or tablets to become lodged in the pharynx or gullet. Also, persons may refuse to swallow creating difficulties for a care provider administering the active compounds in capsule or tablet dosage forms. Other dosage forms also have drawbacks for certain age groups, particularly the aged and children, such as powders and granules which require large volumes of water and syrups and the like which may be difficult to measure for appropriate dosage and which may be expelled rather than swallowed.

Rapidly disintegrating tablets can be administered intrabuccally and are a means to deliver biological actives to the general population, particularly to those in the age groups that have difficulty with other types of dosage forms. Rapid disintegration precludes the need for chewing a capsule or tablet or the need to swallow the capsule or tablet whole in order to deliver the biological active to the patient. Rapidly disintegrating dosage forms also alleviate the drawbacks with other preparations, such as syrups, because liquid measurements are not necessary and large amounts of water required for powder or granule preparations are not needed. Also, because the biological active is delivered through a rapidly disintegrating tablet, there is less opportunity for a person to reject the medication by expelling the dose, including the biological active, from the oral cavity.

Rapidly disintegrating tablets comprising disintegrating or swelling agents to facilitate disintegration in the buccal cavity are described in U.S. Pat. No. 5,464,632. Certain alkali metal salts of carboxymethylcellulose are identified as disintegrating agents for rapidly dissolving tablets in U.S. Pat. No. 3,679,794. Compressed moldings comprising a combination of low moldability saccharides having high dissolution with high moldability saccahrides to allow compression molding while maintaining high dissolution rate are the subject of U.S. Pat. No. 5,576,014. Fast dissolving compression molded tablets comprising an active ingredient, carbohydrates and a barely sufficient amount of water to moisten the surface of the particles, with optional disintegrators, are described in U.S. Pat. No. 5,501,861.

European Patent Specification 0 636 364 B1 concerns compressed pharmaceutical dosage forms said to be rapidly disintegrating containing at least one pharmaceutical active with a taste-masking coating, water-disintegratable compressible carbohydrate and a binder. The taste-masking coating materials comprise cellulose, vinyl pyridine styrene, acrylates and their acids; or combinations of these and not lipid-based materials.

It was an object of the invention to develop novel compressed tablets that will rapidly disintegrate when administered in the buccal cavity, e.g the mouth.

It was a further object of the invention to develop rapidly disintegrable compressed tablets having a biological active comprising a lipid-based taste-masking, sustained release, controlled release and/or barrier/protective coating.

It was another object of the invention to develop rapidly disintegrable compressed tablets characterized as having improved taste, sustained release, controlled release, acid protection and reduced metabolic or chemical denaturation of the biologically active compound.

These and other objects of the invention have been achieved by the compressed tablets described herein comprising at least one biologically active compound, preferably having biologically active materials with a lipid-based taste-masking, sustained release, controlled release and/or barrier/protective coating, one or more bulking agents having a surface area to volume ratio greater than about 1.0 cm⁻¹, one or more binders and one or more lubricants. The compressed tablets will disintegrate within about 40 seconds, or less, preferably within about 20 seconds, or less, of being exposed to saliva in the mouth.

In the present Specification, all parts and percentages are by weight/weight unless otherwise specified.

### SUMMARY OF THE INVENTION

The invention involves a rapidly disintegrating compressed tablet comprising from about 20% to about 98.5% of one or more bulking agents having a surface area to volume ratio greater than about 1.0 cm⁻¹, about 1% to about 15% of one or more binders, about 0.5% to about 3% of one or more lubricants and up to about 60% of one or more biologically active compounds. The tablets may further comprise fillers, additives and other excipients. The compressed tablets are made by dry blending the ingredients using dried compression blend processing on a standard tablet press. The compressed tablets will disintegrate when exposed to water or saliva in the mouth within about 40 seconds, or less, preferably within about 20 seconds, or less.

The biologically active compound preferably has a nominal size less than about 375 microns. Secondly, the most preferred biologically active compound of the invention comprises biologically active material coated with a lipid-based material. In coated form, the biological active compound comprises from about 80% to about 99% of biological active material and about 1% to about 20% lipid-based coating material, or mixtures of such materials, having a melting point greater than about 45° C.

The rapidly disintegrating compressed tablets are administered to human and animal patients via the mouth. A method for delivery of biological active comprises providing the rapidly disintegrating compressed tablet of the invention and placing the tablet in the mouth of the patient.

### DESCRIPTION OF THE DRAWING

FIG. 1 is a representation as a function of the time of the dissolution profile of uncoated acetaminophen and acetaminophen having 4.6% lipid-based coating according to the invention.

### DETAILED DESCRIPTION OF THE INVENTION

The invention pertains to rapidly disintegrating compressed tablets comprising from about 20% to about 98.5%, preferably about 60% to about 80%, of one or more bulking agents having a surface area to volume ratio of greater than about 1.0 cm⁻¹, from about 1% to about 15%, preferably about 3% to about 10%, of one or more binders, from about 0.5% to about 3%, preferably about 1% to about 2% , of one or more lubricants and up to about 60%, preferably from about 1% to about 40%, of one or more biologically active compounds. In a preferred embodiment of the invention, the biologically active compounds have biologically active materials of a nominal size of up to about 375 microns, most preferably these biologically active compounds comprise biologically active materials coated with lipid-based materials. The biologically active compounds having a lipid-based coating comprise from about 80% to about 99%, preferably about 88% to about 97%, biologically active material and about 1% to about 20%, preferably about 3% to about 12%, lipid-based coating materials. The rapidly disintegrating, compressed tablets will disintegrate when administered to the mouth or biological fluids or in other aqueous-based fluid.

The bulking agent must have a surface area to volume ratio greater than about 1.0 cm⁻¹. Bulking agents with less surface area to volume ratio are disadvantageous to the objective of the invention as surface area to volume ratio of less than about 1.0 cm⁻¹ increases the disintegration time while decreasing the ability of the components to form a compressed tablet. Any pharmaceutically acceptable bulking agent, or combinations thereof, may be used in the rapidly disintegrating, compressed tablets provided that the bulking agent has the necessary surface area to volume ratio. Preferred bulking agents are polyols, such as those selected from the group consisting of mannitol, sorbitol, xylitol, maltitol and sucrose, and the like, and combinations thereof. Particularly preferred is a spray-dried form of mannitol, available from Roquette Freres Corporation France, Lestrem, France under the trademark PEARLITOL® SD.

Any pharmaceutically acceptable binder, or combinations thereof, may be used in the rapidly disintegrating compressed tablets. Preferred binders are those selected from the group consisting of starches, starch derivatives, celluloses, cellulose derivatives, dextrins, gelatins, gums, poly(ethylene oxides), poly(ethylene glycols), poly(vinyl pyrrolidones), glucose and the like and combinations thereof. Particularly preferred binders are starches, sodium starch glycolate and hydroxy propyl cellulose.

Any pharmaceutically acceptable lubricant, or combinations thereof, may be used in the rapidly disintegrating compressed tablets. Preferred lubricants are those selected from the group consisting of stearic acid, salts of stearic acid, esterified fatty acid glycerides, silicon dioxide, talc, sodium stearyl fumarate, poly(ethylene glycols) and the like, and combinations thereof. Particularly preferred lubricants are magnesium stearate and glyceral behenate.

The biologically active compounds may be a single biologically active material or comprise one or more biologically active materials. The biologically active materials useful for the invention are those selected from the group consisting of analgesics, antibiotics, anticonvulsants, antidiabetics, antidotes, antihistamines, anti-infectives, anti-inflammatories, antineoplastics, antiparkinsonian agents, antipsychotics, antirheumatics, antivirals, appetite suppressants, biological response modifiers, blood modifiers, cardioprotestive agents, cardiovascular agents, central nervous system stimulants, cerebral metabolic enhancers, cholesterol reducers, contraceptives, deodorants, dopamine receptor agonists, erectile dysfunctional agents, fertility agents, galactorrhea inhibitors, gastrointestinal agents, gout agents, including nonsteroidal anti-inflammatory agents, homeopathic agents, hormones, hyper- and hypocalcemia agents, hypnotics, immunodilators, immunosuppressives, migraine agents, minerals, motion sickness agents, muscle relaxants, narcotics, nucleosides, nutritional agents, ophthalmic agents, osteoporosis agents, oxytocics, parasympatholytics, parasympathomimetics, patent ductus arteriosus agents, porphyria agents, prostaglandins, psychotherapeutics, salts, sedatives, smoking cessation agents, sympatholytics, triglyceride reducers, urinary tract agents, uterine relaxants, vasodilators, vitamins and vertigo agents, and the like, and combinations thereof.

In a preferred embodiment of the invention, the biologically active compounds have a nominal size of up to about 375 microns, preferably from about 100 microns to about 250 microns, and may comprise lipid-based coating materials having a melting point of at least about 45° C. This temperature inhibits disintegration of certain biologically active materials at compression temperatures and is greater than human body temperature, which facilitates the sustained and controlled release of the biologically active material. The lipid-based coating materials are selected from the group consisting of ethoxylated fatty acids, ethoxylated fatty alcohols, poly(ethylene oxide) block copolymers, poly(ethylene glycols), esterified fatty acid glycerides, macrogol glycerides, polyglyceryl fatty acids, cellulose derivatives, such as ethyl cellulose, and the like, and combinations thereof. The most preferred are glycerol palmitostearate and glycerol behenate such as that available from the assignee of the invention under the trade marks PRECIROL™ ATO 5 and COMPITROL™ 888 ATO, respectively.

The rapidly disintegrating compressed tablets may also comprise additives, fillers and other excipients. The term “excipient(s)”, as used herein, means, one or more compatible solid or liquid filler diluents or encapsulating substances, which are suitable for oral administration to a human and encompasses all of the ingredients of the pharmaceutical compositions except the biologically active compounds or material. The term “compatible”, as used herein, means that the components of the compositions of the invention are capable of being commingled with the biologically active compounds or materials and with each other, in a manner such that there is no interaction, which would substantially reduce the efficacy of the compositions under ordinary use situations. Excipients must, of course, be of sufficiently high purity and sufficiently low toxicity to render them suitable for administration to the human being.

Examples of additives and fillers include flavoring agents such as those selected from the group consisting of oil of peppermint, oil of wintergreen, oil of spearmint, clove bud oil, parsley oil, eucalyptus oil, menthol, menthane, anethole, methyl salicylate, eucalyptol, cassia, 1-methyl acetate, sage, eugenol, oxanone, alpha-irisone, marjoram, lemon, orange, propenyl guaethol acetyl, cinnamon, vanilla, thymol, linalool, cinnamaldehyde glycerol acetal, licorice extracts and the like, and combinations thereof; coloring agents and/or dyes such as Food, Drug and Cosmetic (FD&C) colorants and cooling agents such as menthol, N-ethyl p-methane-3-carboxamide, 3,1-methoxy propane 1,2-diol and the like, and combinations thereof.

Examples of other excipients that may be a component of the rapidly disintegrating compressed tablets include sugars, such as lactose, glucose and sucrose; powdered tragacanth; malt; gelatin; talc; vegetable oils such as peanut oil, cottonseed oil, sesame oil, olive oil, corn oil and oil of theobroma; agar and alginic acid; wetting agents; sweetening agents (including nonnutritive sweeteners such as aspartame and saccharine); tableting agents; stabilizers; antioxidants; warming agents; numbing agents, and preservatives as well as other non-toxic compatible substances used in pharmaceutical formulations.

The rapidly disintegrating compressed tablets are administered to provide a predetermined dose of biologically active compounds through oral ingestion. The tablets will typically disintegrate within about 40 seconds, or less, preferably within about 20 seconds, or less, of being exposed to saliva in the mouth. Thus, biologically active compounds can be delivered to a patient without the need for chewing the tablet or requiring that the tablet be swallowed whole. These types of dosage forms are particularly suited for oral administration of active compounds to the elderly and young.

The method for delivering biologically active compounds to a human or animal patient via the rapidly disintegrating compressed tablets described herein comprises the steps of providing one or more of the rapidly disintegrating compressed tablets and placing the tablet in the buccal cavity of the patient. The compressed tablet will disintegrate within about 40 seconds, or less, preferably within about 20 seconds, or less, after being placed in the buccal cavity.

The rapidly disintegrating compressed tablets are characterized as having improved taste, sustained release, controlled release, acid protection and reduced metabolic or chemical denaturation of the biologically active compound. In particular, the pharmaceutical compositions comprising biologically active materials and the lipid-based coating have, in addition to the improved physical and chemical properties discussed above, enhanced properties due in part to the coating, compared to other compositions, such as non-coated active ingredients. These properties include better chemical stability, improved taste as a result of the taste-masking properties of the coating, better sustained and controlled release of active compound, better acid protection, more reduced metabolic or chemical denaturation, improved flow, odor masking and increased compressibility of the biologically active compound.

### EXAMPLES

### Example 1

A dry blend of the following components were mixed in a 5 liter V-Blender in the sequence specified below to obtain micro, rapidly disintegrating, tablet placebos.

| | | |
|---|---|---|
| Combine | | |
| PEARLITOL SD 200 | 1.33 Kg | (Bulking agent) |
| Starch, USP | 0.30 Kg | (Binder) |
| Blend for 5 minutes | | |
| Add additional | | |
| PEARLITOL SD 200 | 1.34 Kg | |
| Blend for 5 minutes | | |
| Add | | |
| Magnesium stearate, USP | 0.03 Kg | (Lubricant) |
| Blend for 3 minutes | | |

PEARLITOL SD 200 is available from Roquette Freres Corporation, France and comprises spray-dried mannitol with a surface area to volume ratio greater than 1.0 cm⁻¹. The starch used was USP grade available from Grain Processing Corporation (“GPC”) Muscatine, Iowa, U.S.A. The blend obtained from the mixing sequence described above was fed to a Korsch PH 106 tablet press from Korsch America, Inc., South Easton, Mass., U.S.A. and compressed to form 0.25 inch tablet placebos weighing 89 mg.

Disintegration of the tablet placebos was evaluated by applying USP testing procedures. The tablet placebos disintegrated in standing water and in the mouth in less than about 10 seconds.

### Example 2

A dry blend of the following components, from the same suppliers as in Example 1, were mixed in a 5 liter V-Blender in the sequence specified below to obtain rapidly disintegrating, tablet placebos.

| | | |
|---|---|---|
| Combine | | |
| PEARLITOL SD 200 | 1.33 Kg | (Bulking agent) |
| Starch, USP | 0.30 Kg | (Binder) |
| Blend for 5 minutes | | |
| Add additional | | |
| PEARLITOL SD 200 | 1.34 Kg | |
| Blend for 5 minutes | | |
| Add | | |
| Magnesium stearate, USP | 0.03 Kg | (Lubricant) |
| Blend for 3 minutes | | |

The blend obtained from the mixing sequence described above was fed to the Korsch PH 106 tablet press and compressed to form 0.4375 inch tablets weighing 325 mg. Disintegration of the tablet placebos was evaluated by applying USP testing procedures, which are incorporated herein by reference. The tablet placebos disintegrated in standing water and the mouth in less than about 10 seconds for tablets compressed to a hardness of less than 3 kp.

### Example 3

This Example concerns rapidly disintegrating, taste-masked, acetaminophen tablets made by combining a bulking agent, binder and lubricant with a biologically active component of lipid-based coated acetaminophen particles.

The coated acetaminophen particles were made by a hot melt fluid bed process. Acetaminophen (i.e. biologically active material) particles of an average size, determined by laser light scattering, of 280 microns were coated with lipid material, glyceryl palmitostearate (PRECIROL™ ATO 5). A hot melt fluid bed process was used to deposit glyceryl palmitostearate on the acetaminophen particle at a level of 4.6% of the final coated particle. The lipid-based coated acetaminophen particles were on average 345 microns determined by laser light scattering. These acetaminophen particles were incorporated into the rapidly disintegrating tablets of this Example in accordance with the mixing procedure specified below.

Prior to incorporating the coated acetaminophen particles into the rapidly disintegrating tablets, however, taste, texture, and dissolution characteristic were evaluated. The coated acetaminophen particles were free flowing and did not exhibit a taste characteristic of acetaminophen when administered in the buccal cavity of the mouth and swallowed. The texture of the particles was not gritty. The dissolution of the acetaminophen from the particle was greated than 80% in 45 minutes.

Dissolution testing of the acetaminophen particles was carried out using USP XXIII “Acetaminophen capsules” and “Dissolution '711”, which are incorporated herein by reference. The method was adapted to use a relevant quantity of powder, i.e. an amount equivalent to 500 mg of acetaminophen in the dissolution bowl. This quantity represents a standard amount of aspirin in marketed U.S.A. over-the-counter (OTC) products. The operating conditions included 900 ml of water at 37° C. in a USP Apparatus 2 with paddle speed set at 50 rpm.

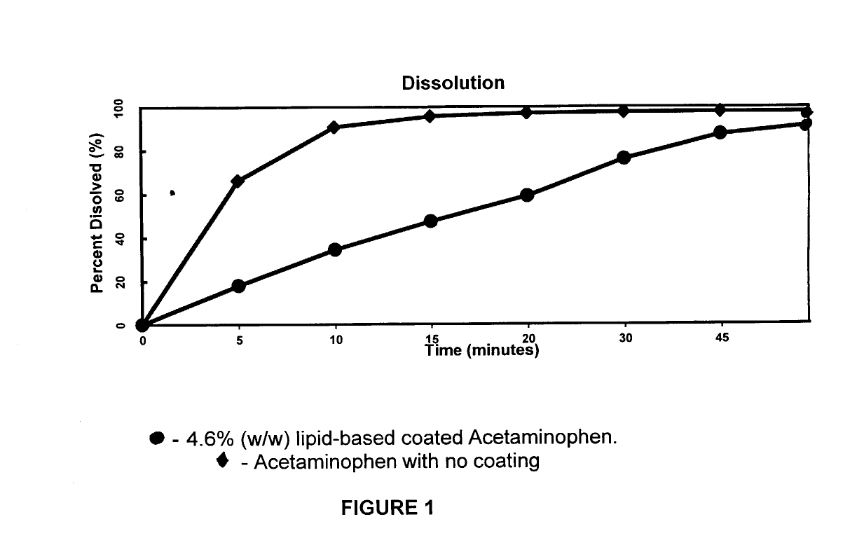

The data relating to dissolution of un-coated and coated acetaminophen are presented in Table 1. FIG. 1 presents the dissolution-curves of acetaminophen and 4.6% lipid-based coated acetaminophen, i.e. 4.6% coating and 95.4% acetaminophen.

TABLE 1
Dissolution Rate (n = 6)

| | Dissolution % | Dissolution (%) /σ (%) |
|---|---|---|
| | 100% | 4.6% Coating/95.4% |
| Time (min) | Acetaminophen | Acetaminophen |
| 5 | 66.3 | 18.0/1.8 |
| 10 | 90.8 | 34.5/2.3 |
| 15 | 95.7 | 47.5/3.7 |
| 20 | 97.1 | 59.1/3.9 |
| 30 | 97.5 | 76.0/4.7 |
| 45 | 97.7 | 87.4/2.9 |
| 60 | 97.7 | 91.7/1.9 |

The rapidly disintegrating compressed tablets were made by first mixing a dry blend of the following components in a 5 liter V-Blender in the sequence specified below to obtain Blend #1.

| | | |
|---|---|---|
| Combine | | |
| PEARLITOL SD 200 | 1.006 Kg | (Bulking agent) |
| Starch, USP (from GPC) | 0.226 Kg | (Binder) |
| Blend for 5 minutes | | |
| Add | | |
| PEARLITOL SD 200 | 1.006 Kg | |
| Blend for 5 minutes to form Blend #1 | | |

Blend #1 (bulking agent and binder) was then mixed in a 5 liter V-Blender with other components in accordance with the sequence specified below to obtain Blend #2.

| | | |
|---|---|---|
| Combine | | |
| Blend #1 | 1.492 Kg | (Bulking agent & binder) |
| Coated Aetaminophen | 0.492 Kg | |
| Blend for 5 minutes | | |
| Add | | |
| Magnesium stearate, USP | 0.015 Kg | (Lubricant) |
| Blend for 3 minutes | | |
| To obtain Blend #2 | | |

Blend #2 was fed to the Korsch PH 106 tablet press and compressed to form 0.4375 inch tablets weighing 330 mg. Disintegration of the tablets comprising coated acetaminophen was evaluated by applying USP testing procedures. The tablets disintegrated in standing water and the mouth in less than about 10 seconds for tablets compressed to a hardness of less than 2 kp.

1. Rapidly disintegrating compressed tablets comprising from about 20% to about 98.5% of one or more bulking agents having a surface area to volume ratio of greater than 1.0 cm⁻¹, from about 1% to about 15% of one or more binders, from about 0.5% to about 3% of one or more lubricants and up to about 60% of one or more biologically active compounds.

2. The tablets of claim 1 where the biologically active compounds have a nominal size of up to about 375 microns.

3. The tablets of claim 1 wherein the biologically active compounds comprise biologically active materials that are coated with lipid-based materials having a melting point of at least about 45° C.

4. The tablets of claim 3 wherein the biologically active compounds comprise from about 80% to about 99% biologically active material and about 1% to about 20% lipid-based thermal plastic coating materials.

5. The tablets of claim 4 wherein the lipid-based materials are selected from the group consisting of ethoxylated fatty acids, ethoxylated fatty alcohols, poly(ethylene oxide) block copolymers, poly(ethylene glycols), esterified fatty acid glycerides, macrogol glycerides, polyglyceryl fatty acids, cellulose derivatives and combinations thereof.

6. The tablets of claim 5 wherein the lipid-based material comprises glycerol palmitostearate, glycol behenate or ethyl cellulose.

7. The tablets of claim 1 wherein the bulking agents are polyols.

8. The tablets of claim 7 wherein the ployols are selected from the group consisting of mannitol, sorbitol, xylitol, maltitol, sucrose and combinations thereof.

9. The tablets of claim 1 wherein the binders are selected from the group consisting of starch, starch derivatives, celluloses, cellulose derivatives, dextrins, gelatins, gums, poly(ethylene oxides), poly(ethylene glycols), poly(vinyl pyrrolidones), glucose and combinations thereof.

10. The tablets of claim 9 wherein the binders comprise sodium starch glycolate or hydroxy propyl cellulose.

11. The tablets of claim 1 wherein the lubricants are selected from the group consisting of stearic acid, salts of stearic acid, esterified fatty acid glycerides, silicon dioxide, talc, sodium stearyl fumarate, poly(ethylene glycols) and combinations thereof.

12. The tablets of claim 11 wherein the lubricants comprise magnesium stearate or glyceral behenate.

13. The tablets of claim 1 wherein the biologically active compounds comprise one or more biologically active materials selected from the group consisting of analgesics, antibiotics, anticonvulsants, antidiabetics, antidotes, antihistamines, anti-infectives, anti-inflammatories, antineoplastics, antiparkinsonian agents, antipsychotics, antirheumatics, antivirals, appetite suppressants, biological response modifiers, blood modifiers, cardioprotestive agents, cardiovascular agents, central nervous system stimulants, cerebral metabolic enhancers, cholesterol reducers, contraceptives, deodorants, dopamine receptor agonists, erectile dysfunctional agents, fertility agents, galactorrhea inhibitors, gastrointestinal agents, gout agents, homeopathic agents, hormones, hyper- and hypocalcemia agents, hypnotics, immunodilators, immunosuppressives, migraine agents, minerals, motion sickness agents, muscle relaxants, narcotics, nucleosides, nutritional agents, ophthalmic agents, osteoporosis agents, oxytocics, parasympatholytics, parasympathomimetics, patent ductus arteriosus agents, porphyria agents, prostaglandins, psychotherapeutics, salts, sedatives, smoking cessation agents, sympatholytics, triglyceride reducers, urinary tract agents, uterine relaxants, vasodilators, vitamins and vertigo agents and combinations thereof.

14. The tablets of claim 1 further comprising flavoring agents selected from the group consisting of oil of peppermint, oil of wintergreen, oil of spearmint, clove bud oil, parsley oil, eucalyptus oil, menthol, menthane, anethole, methyl salicylate, eucalyptol, cassia, 1-methyl acetate, sage, eugenol, oxanone, alpha-irisone, marjoram, lemon, orange, propenyl guaethol acetyl, cinnamon, vanilla, thymol, linalool, cinnamaldehyde glycerol acetal, licorice extracts and combinations thereof.

15. The tablets of claim 1 further comprising coloring agents and/or dyes.

16. The tablets of claim 1 further comprising cooling agents selected from the group consisting of menthol, N-ethyl p-methane-3-carboxamide, 3,1-methoxy propane 1,2-diol and combinations thereof.

17. The tablets of claim 1 further comprising excipients selected from the group consisting of sugars, powdered tragacanth, malt, gelatin, talc, vegetable oils, agar, alginic acid, wetting agents, sweetening agents, tableting agents, stabilizers, antioxidants, warming agents, numbing agents, preservatives and combinations thereof.

18. A method of delivering biologically active compounds to a human or animal patient comprising the steps of providing one or more rapidly disintegrating compressed tablets having from about 20% to about 98.5% of one or more bulking agents having a surface area to volume ratio of greater than 1.0 cm⁻¹, from about 1% to about 15% of one or more binders, from about 0.5% to about 3% of one or more lubricants up to about 60% of one or more biologically active compounds and optionally, flavoring agents, coloring agents, cooling agents and other excipients, and placing the tablet in the buccal cavity wherein the compressed tablet disintegrates within about 40 seconds, or less, after being placed in the buccal cavity.

19. The method of claim 18 wherein the one or more biologically active compounds comprise from about 80% to about 99% biologically active materials and about 1% to about 20% lipid-based thermal plastic coating materials having a melting point of at least about 45° C.

20. The method of claim 19 wherein the coating materials are selected from the group consisting of ethoxylated fatty acids, ethoxylated fatty alcohols, poly(ethylene oxide) block copolymers, poly(ethylene glycols), esterified fatty acid glycerides, macrogol glycerides, polyglyceryl fatty acids, cellulose derivatives and combinations thereof.

21. Rapidly disintegrating compressed tablets comprising from about 20% to about 98.5% of one or more bulking agents having a surface area to volume ratio of greater than 1.0 cm⁻¹, from about 1% to about 15% of one or more binders, from about 0.5% to about 3% of one or more lubricants and from about 1% to about 60% of one or more biologically active compounds having biologically active materials that are coated with lipid-based materials having a melting point of at least about 45° C.

22. The tablets of claim 21 wherein the biologically active compounds have a nominal size of up to about 375 microns.

23. The tablets of claim 21 wherein the lipid-based material comprises glycerol palmitostearate, glycerol behenate or ethyl cellulose.
